(19) United States
(12) Patent Application Publication
(10) Pub. No.: US 2017/0157013 A1
(43) Pub. Date: Jun. 8, 2017

(54) Metal Amino Acid Complexes for Bacterial Aggregation

(71) Applicant: Colgate-Palmolive Company, New York, NY (US)

(72) Inventors: Lyndsay Schaeffer-Korbylo, Flemington, NJ (US); Lisa Manus, Plainsboro, NJ (US)

(73) Assignee: Colgate-Palmolive Company, New York, NY (US)

(21) Appl. No.: 14/960,960

(22) Filed: Dec. 7, 2015

(51) Int. Cl. A61K 8/58; A61Q 11/00

(52) U.S. Cl. CPC A61K 8/58; A61Q 11/00; A61K 2800/58

(57) ABSTRACT

Disclosed herein are methods of promoting the aggregation and/or immune clearance of oral bacteria using oral care compositions comprising metal-amino acid complexes.

### BACKGROUND

Complexes between metal ions and amino acids are known. Some of these, especially complexes between divalent metal ions and basic amino acids, have seen use in the field of oral care for their ability to treat dentinal hypersensitivity. Certain complexes, such as zinc-bis(lysine)-halide, zinc-bis(arginine)-halide and zinc-(trimethylglycine)halide, have been discovered to form stable, homogenous aqueous solutions, which under certain conditions, can precipitate zinc hydroxide, zinc oxide and other zinc species. The precipitation of such insoluble salts enables oral care compositions comprising these complexes to effectively plug the dentinal tubules of the teeth that transmit sensations of hypersensitivity.

Salts of divalent metal ions, especially zinc, stannous, and copper, are generally known as antibacterial agents. Zinc and stannous salts especially have been used in oral care compositions for their anti-plaque, anti-tarter and anti-malodor effects, which stem from the enhanced bioavailability of ligated zinc ions to interact with oral surfaces. Examples of zinc complexes used in the past include zinc oxide, zinc citrate and zinc chloride. The efficacy and tolerability of these salts have varied due to perceived astringency, cosmetic appearance and interactions between ingredients in these formulations.

The aggregation of bacteria within the human (or other animal) body is an important signal that the immune system uses to promote clearance of harmful bacteria. While individual bacterial cells are difficult for the immune system to detect and clear, larger aggregates are much more efficiently targeted for removal and cleared. Indeed, one important mechanism by which antibodies operate is by causing the aggregation of bacteria, thus flagging them for ingestion by passing phagocytic immune cells. Various molecules, both natural and synthetic, have been described as promoting bacterial aggregation, and can thus exert a beneficial antibacterial effect in the human body.

Free metal ions, including zinc ions, have been demonstrated to promote bacterial aggregation, but this is thought to reflect a non-specific effect on biological membranes. Indeed, this effect has been demonstrated for some eukaryotic cells as well.

While the prior art discloses the use of various metal amino acid complexes in oral compositions, and the use of various metal ions for the aggregation of bacteria, there is still a need for additional compositions and methods which provide selective bacterial aggregation that could be of benefit in oral care compositions.

### BRIEF SUMMARY

It has now been discovered that certain divalent metal ions can form highly soluble complexes with amino acids, and that these complexes can effectively and selectively promote the aggregation of bacteria. Preferred complexes are formed between zinc(II) ions or copper(II) ions and amino acids, especially basic amino acids (e.g., lysine or arginine).

In some embodiments, the complex comprising zinc and amino acid and optionally an anion and/or oxygen, forms a soluble cationic moiety, which in turn may form a salt with a halide or other anion. While promoting improved bacterial aggregation in comparison to formulations with insoluble zinc salts, the formulations comprising the zinc-amino acid complex do not exhibit the poor taste and mouthfeel, poor fluoride delivery, and poor foaming and cleaning associated with conventional zinc-based oral care products using soluble zinc salts.

In one particular embodiment, the zinc-amino acid complex is a zinc-lysine-HCl complex, for example, the complex designated ZLC, which may be formed from a mixture of zinc oxide and lysine hydrochloride. ZLC has the chemical structure [Zn(C₆H₁₄N₂O₂)₂Cl]⁺Cl⁻, and may exist in solution of the cationic cation ([Zn(C₆H₁₄N₂O₂)₂Cl]⁺) and the chloride anion, or may be a solid salt, e.g., a crystal, optionally in mono- or dihydrate form. This ZLC complex has been disclosed in WO 2014/098822, WO 2014/098824, WO 2014/098825, and WO 2014/098826, the contents of each of which are hereby incorporated by reference in their entireties.

The present disclosure thus provides oral care compositions, for example mouthwash, oral gel or dentifrice compositions, which comprise a metal-amino acid complex, e.g. a zinc-amino acid complex, e.g., a zinc-lysine-chloride complex, e.g., ZLC, for use in promoting the aggregation of oral bacteria. The compositions may optionally further comprise a fluoride source and or an additional phosphate source. The compositions may be formulated in a suitable oral care formulation e.g., a conventional dentifrice, oral gel or mouthwash base, e.g., comprising one or more abrasives, surfactants, foaming agents, vitamins, polymers, enzymes, humectants, thickeners, antimicrobial agents, preservatives, flavorings, and/or colorants.

The present disclosure further provides methods of using oral care compositions comprising a metal-amino acid complex, e.g., a zinc-amino acid complex, e.g., a zinc-lysine-chloride complex (ZLC), to promote the aggregation of and immune clearance of oral bacteria. This can result in multiple favorable effects on oral hygiene and oral health, including reduction and inhibition of acid erosion of the enamel, cleaning of the teeth, reduction of bacterially-generated biofilm and plaque, reduction of gingivitis, inhibition of tooth decay and the formation of cavities. The method comprises the application of a composition of the present disclosure to the teeth.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

The present disclosure provides, in a first embodiment, a method (Method 1) of promoting the aggregation and/or immune clearance of oral bacteria, comprising administering to the oral cavity of a person an oral care composition (Composition 1) comprising a metal-amino acid complex. In some embodiments. Method 1 is effective in treating diseases, disorders and conditions of the oral cavity, such as gingivitis, periodontitis, halitosis, cavity formation, enamel erosion, and oral infection (e.g., oral candidiasis), or to disrupt the formation of dental plaque and bacterial biofilm.

In a second embodiment, the present disclosure provides a method (Method 2) of treating a disease, disorder or condition of the oral cavity, comprising the step of administering to a patient in need thereof an oral care composition (Composition 1) comprising a metal-amino acid complex to promote the aggregation and/or immune clearance of oral bacteria. In specific embodiments, said patient suffers from a disease, disorder or condition of the oral cavity, such as gingivitis, periodontitis, halitosis, cavity formation, enamel erosion, and/or oral infection (e.g., oral candidiasis).

In particular embodiments, Method 1 or Method 2 comprises the administration of an oral care composition, wherein the oral care composition is:
1.1. Composition 1, wherein the metal-amino acid complex comprises a divalent metal ion (e.g., zinc(II) or copper(II)).
1.2. Composition 1 or 1.1, wherein the metal-amino acid complex comprises zinc(II) ion.
1.3. Composition 1 or 1.1, wherein the metal-amino acid complex comprises copper(II) ion.
1.4. Composition 1 or 1.1-1.3, wherein the amino acid is a natural amino acid in the L- or D-form, or a modified amino acid in the L- or D-form (e.g., an N-alkylated amino acid, e.g., a mono-, di-, or tri-N-alkylated amino acid).
1.5. Composition 1 or 1.1-1.4, wherein the amino acid is selected from lysine, glycine, tetramethylglycine (TMG) and arginine, in free or orally acceptable acid addition salt form, e.g., hydrochloride form.
1.6. Composition 1 or 1.1-1.5 wherein the amino acid is a basic amino acid, e.g., arginine or lysine, in free or orally acceptable salt form.
1.7. Any of the foregoing compositions further comprising a halide (e.g., chloride, bromide or fluoride) in ionic association with the metal ion and amino acid.
1.8. Any of the foregoing compositions wherein the molar ratio of metal ion:amino acid is from 3:1 to 1:5, e.g., about 1:2 and the molar ratio of metal ion:halide where present is from 3:1 to 1:3, e.g., about 1:2.
1.9. Any of the foregoing compositions wherein the metal ion-amino acid complex is formed, in whole or in part, in situ after the composition is applied.
1.10. Any of the foregoing compositions wherein the metal ion-amino acid complex is formed, in whole or in part, in situ after the composition is formulated.
1.11. Any of the foregoing compositions, wherein the amino acid is lysine.
1.12. Any of the foregoing compositions, wherein the metal ion-amino acid complex is a zinc-lysine complex.
1.13. Any of the foregoing compositions, wherein the metal ion (e.g. zinc) is present in an amount of 0.05 to 10% by weight of the composition, optionally at least 0.1, at least 0.2, at least 0.3, at least 0.4, at least 0.5, at least 1, at least 2, at least 3, or at least 4 up to 10% by weight of the composition, e.g. about 1-3%, e.g., about 2-2.7% by weight.
1.14. Any of the foregoing compositions, wherein the amino acid is present in an amount of 0.05 to 30% by weight of the composition, optionally at least 0.1, at least 0.2, at least 0.3, at least 0.4, at least 0.5, at least 1, at least 2, at least 3, at least 4, at least 5, at least 10, at least 15, at least 20 up to 30% by weight, e.g., about 1-10% by weight.
1.15. Any of the foregoing compositions, wherein a molar ratio of zinc to amino acid is 2:1 to 1:4, optionally 1:1 to 1:4, 1:2 to 1:4, 1:3 to 1:4, 2:1 to 1:3, 2:1 to 1:2, or 2:1 to 1:1, e.g., about 1:2 or 1:3
1.16. Any of the foregoing compositions comprising a halide in ionic association with zinc and amino acid, wherein the halide is selected from the group consisting of fluorine, chlorine, bromine and mixtures thereof.
1.17. Any of the foregoing compositions wherein the zinc amino acid complex is a zinc lysine chloride complex (e.g., (ZnLys₂Cl)⁺Cl⁻ or (ZnLys₃)²⁺Cl₂) or a zinc arginine chloride complex.
1.18. Any of the foregoing compositions, wherein the zinc amino acid complex is a zinc lysine chloride complex, e.g., ZLC, e.g., a zinc lysine chloride complex having the chemical structure [Zn(C₆H₁₄N₂O₂)₂Cl]⁺Cl⁻, either in solution of the cationic cation (e.g., [Zn(C₆H₁₄N₂O₂)₂Cl]⁺) and the chloride anion, or in solid salt form, e.g., crystal form, optionally in mono- or dihydrate form.
1.19. Any of the foregoing compositions in the form of a toothpaste, gel, mouthwash, powder, cream, strip (e.g., thin films), or gum.
1.20. Any of the foregoing compositions in an orally acceptable base, e.g., a mouthwash, gel, or dentifrice base.
1.21. Any of the foregoing compositions in the form of a dentifrice, e.g., wherein the metal-amino acid complex (e.g., zinc-lysine, zinc-arginine or copper-lysine complex) is present in an effective amount, e.g., in an amount of 0.5-4% by weight of metal (e.g., zinc), e.g., about 1-3% by weight of metal (e.g., zinc), in a dentifrice base.
1.22. Any of the foregoing compositions in the form of a dentifrice, wherein the dentifrice base comprises an abrasive, e.g., an effective amount of a silica abrasive, e.g., 10-30%, e.g., about 20%.
1.23. Any of the foregoing compositions in the form of a mouthwash, e.g., wherein the metal-amino acid complex (e.g., zinc-lysine, zinc-arginine or copper-lysine complex) is present in an effective amount, e.g., in an amount of 0.1 to 2% by weight of the composition, or 0.5 to 1.5%, or 1-1.3% by weight of the composition, or wherein the metal-amino acid complex is present in an amount to provide 0.01-0.5% zinc by weight of the composition, e.g., 0.01-0.25%, or 0.014-0.14% zinc by weight of the composition.
1.24. Any of the foregoing compositions further comprising an effective amount of a fluoride ion source, e.g., providing 50 to 3000 ppm fluoride.
1.25. Any of the foregoing compositions further comprising an effective amount of fluoride, e.g., wherein the fluoride is a salt selected from stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof.
1.26. Any of the preceding compositions comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these, e.g., in an amount of 1-20%, e.g., 2-8%, e.g., ca. 5%, by weight of the composition.
1.27. Any of the foregoing compositions comprising buffering agents, e.g., phosphate buffers or citrate buffers, for example, sodium phosphate buffer (e.g., sodium phosphate monobasic, disodium phosphate and/or phosphoric acid).
1.28. Any of the foregoing compositions comprising a humectant, e.g., selected from glycerin, sorbitol, propylene glycol, polyethylene glycol, xylitol, and mixtures thereof, e.g. comprising at least 20%, e.g., 20-40%, e.g., 25-35% glycerin.
1.29. Any of the preceding compositions comprising one or more surfactants, e.g., selected from anionic, cationic, zwitterionic, and nonionic surfactants, and mixtures thereof, e.g., comprising an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2% cocamidopropylbetaine.
1.30. Any of the preceding compositions further comprising a viscosity modifying amount of one or more of polysaccharide gums, for example xanthan gum or carrageenan, silica thickener, and combinations thereof.
1.31. Any of the preceding compositions comprising gum strips or fragments.
1.32. Any of the preceding compositions further comprising flavoring, fragrance and/or coloring.
1.33. Any of the foregoing compositions comprising an effective amount of one or more antibacterial agents, for example comprising an antibacterial agent selected from halogenated diphenyl ether (e.g. triclosan), herbal extracts and essential oils (e.g., rosemary extract, tea extract, magnolia extract, magnolol, honokiol, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing; e.g., comprising triclosan, or cetylpyridinium chloride or magnolol or honokiol.
1.34. Any of the preceding compositions further comprising a whitening agent, e.g., a selected from the group consisting of peroxides, metal chlorites, perborates, percarbonates, peroxyacids, hypochlorites, and combinations thereof.
1.35. Any of the preceding compositions further comprising hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate);
1.36. Any of the preceding compositions, further comprising an agent that interferes with or prevents bacterial attachment, e.g., solbrol or chitosan.
1.37. Any of the preceding compositions, further comprising a source of calcium and phosphate selected from (i) calcium-glass complexes, e.g., calcium sodium phosphosilicates, and (ii) calcium-protein complexes, e.g., casein phosphopeptide-amorphous calcium phosphate
1.38. Any of the preceding compositions, further comprising a soluble calcium salt, e.g., selected from calcium sulfate, calcium chloride, calcium nitrate, calcium acetate, calcium lactate, and combinations thereof.
1.39. Any of the preceding compositions further comprising a physiologically or orally acceptable potassium salt, e.g., potassium nitrate or potassium chloride, in an amount effective to reduce dentinal sensitivity.
1.40. Any of the foregoing compositions further comprising an anionic polymer, e.g., a synthetic anionic polymeric polycarboxylate, e.g., wherein the anionic polymer is selected from 1:4 to 4:1 copolymers of maleic anhydride or acid with another polymerizable ethylenically unsaturated monomer, e.g., wherein the anionic polymer is a methyl vinyl ether/maleic anhydride (PVM/MA) copolymer having an average molecular weight (M.W.) of about 30,000 to about 1,000,000, e.g. about 300,000 to about 800,000, e.g., wherein the anionic polymer is about 1-5%, e.g., about 2%, of the weight of the composition.
1.41. Any of the foregoing compositions, further comprising a non-ionic polymer, e.g. polyvinylpyrrolidone (PVP), for example linear or cross-linked PVP.
1.42. Any of the preceding compositions further comprising a breath freshener, fragrance or flavoring.
1.43. Any of the foregoing compositions, wherein the pH of the composition is approximately neutral, e.g., from pH 6 to pH 8 e.g., about pH 7.
1.44. Any of the foregoing compositions in the form of a mouthwash wherein the amino acid is lysine and the zinc and lysine form a zinc-lysine-chloride complex having the chemical structure [Zn(CH₁₄N₂O₂)₂Cl]⁺Cl⁻, in an amount to provide 0.5-2%, e.g., about 1% zinc by weight, the composition further comprising humectant, e.g., sorbitol, propylene glycol and mixtures thereof, e.g., in an amount of 10-25%, e.g., about 15-20%, non-ionic surfactant, e.g., poloxamer, e.g., in an amount of 0.1-1%, and sweetener, flavorings, and water, e.g., a mouthwash comprising

| Ingredients | Wt % |
|---|---|
| Sorbitol | 3-7%, e.g., about 4% |
| ZLC | to provide 0.01-0.5% zinc e.g., |
| | 0.01-0.25%, e.g., about 0.08% |
| Propylene Glycol | 5-10%, e.g., about 7% |
| Surfactant (e.g., Poloxamer, | 0.1-1%, e.g., about 0.4% |
| Poloxomer 407, or CAP betaine | |
| Glycerin | 5-10%, e.g., about 7.5% |
| Flavor and/or sweetener | 0.01-1%, e.g., about 0.1% |
| Deionized water | 70-85%, e.g., about 80% |

1.45. Any of the foregoing compositions in the form of an oral gel, wherein the amino acid is lysine and the zinc and lysine form a zinc-lysine-chloride complex having the chemical structure [Zn(C₆H₁₄N₂O₂)₂Cl]⁺Cl⁻, in an amount to provide 0.1-2%, e.g., about 0.5% zinc by weight, and further comprising humectant, e.g., sorbitol, propylene glycol and mixtures thereof, e.g., in an amount of 45-65%, e.g., about 50-60%, thickeners, e.g., cellulose derivatives, e.g., selected from carboxymethyl cellulose (CMC), trimethyl cellulose (TMC) and mixtures thereof, e.g., in an amount of 0.1-2%, sweetener and/or flavorings, and water, e.g., an oral gel comprising

| Ingredients | Wt % |
|---|---|
| Sorbitol | 40-60%, e.g., 50-55% |
| ZLC | to provide 0.1-2% Zn, |
| | e.g about 0.5% Zn |
| Carboxymethyl cellulose (CMC) and | 0.5-1%, e.g., about 0.7% |
| Trimethyl cellulose (TMC) | |
| Flavoring and/or sweetener | 0.01-1% |
| Propylene Glycol | 1-5%, e.g., about 3.00% |

In some embodiments, the oral bacteria aggregated by the uses and methods disclosed herein include one or more of A. viscous, L. casei, S. oralis, V. parvula and F. nucleatum. In some embodiments, the uses and methods disclosed herein are effective to aggregate Gram negative oral bacteria. In some embodiments, the patients administered the compositions according to Method 1 or Method 2 have demonstrated one or more of A. viscous, L. casei, S. oralis, V. parvula and F. nucleatum present in their oral cavities. In some embodiments, the patients administered the compositions according to Method 1 or Method 2 have been diagnosed with an oral bacterial disease or disorder, e.g., an oral bacterial infection caused by one or more of A. viscous, L. casei, S. oralis, V. parvula and F. nucleatum. In some embodiments, the patients administered the compositions according to Method 1 or 2 are suffering from conditions or recovering from treatments that predispose them to oral bacterial infections (e.g., periodontitis, gingivitis, oral surgery, tooth extraction, root canal treatment), such as infection by Gram negative bacteria, or by one or more of A. viscous, L. casei, S. oralis, V. parvula and F. nucleatum.

In some embodiments, Method 1 or 2 further provides effectiveness to reduce and inhibit acid erosion of the enamel, clean the teeth, reduce bacterially-generated biofilm and plaque, reduce gingivitis, inhibit tooth decay and formation of cavities, and reduce dentinal hypersensitivity. In one aspect, Method 1 or 2 is used in conjunction with a regimen of tooth brushing, such that brushing dislodges oral bacteria from the structures of the oral cavity (e.g., the teeth and gums) and the resulting the present Methods assist in the aggregation and clearance of these dislodged and free-floating bacteria. In some embodiments, the Methods allow for the aggregation and destruction of oral bacteria transiently released from the oral surfaces (e.g., the bacterial biofilm of the teeth) before they can reattach to solid structures in the oral cavity.

In some embodiments, the present disclosure provides a method according to Method 1 or 2, which is effective for one or more of the following: (i) to reduce hypersensitivity of the teeth, (ii) to reduce plaque accumulation, (iii) to reduce or inhibit demineralization and promote remineralization of the teeth, (iv) to inhibit microbial biofilm formation in the oral cavity, (v) to reduce or inhibit gingivitis, (vi) to promote healing of sores or cuts in the mouth, (vii) reduce levels of acid producing bacteria, (viii) to increase relative levels of non-cariogenic and/or non-plaque forming bacteria, (ix) to reduce or inhibit formation of dental caries, (x) to reduce, repair or inhibit pre-carious lesions of the enamel, e.g., as detected by quantitative light-induced fluorescence (QLF) or electrical caries measurement (ECM), (xi) to treat, relieve or reduce dry mouth, (xii) to clean the teeth and oral cavity, (xiii) to reduce erosion, (xiv) to whiten teeth; (xv) to reduce tartar build-up, (xvi) to freshen the breath and/or treat or prevent halitosis, and/or (xvii) to promote systemic health, including cardiovascular health, e.g., by reducing the potential for systemic infection via the oral tissues, the method comprising applying any of Compositions 1, et seq. as described above to the oral cavity of a person in need thereof, e.g., one or more times per day. The present disclosure further provides Compositions 1, et seq. for use in any of these methods.

The present disclosure further provides the use an oral care composition (Use 1) comprising a metal-amino acid complex (e.g., Composition 1, et seq.) to promote the aggregation of oral bacteria and/or to promote the immune clearance of oral bacteria. In some embodiments, the use of Composition 1, et seq., to promote the aggregation and/or immune clearance of oral bacteria is effective in treating diseases, disorders or conditions of the oral cavity (e.g., gingivitis, periodontitis, halitosis, cavity formation, enamel erosion, or oral infection) or in disrupting the formation of plaque and bacterial biofilm. In specific embodiments, said use is effective to reduce and inhibit acid erosion of the enamel, clean the teeth, reduce bacterially-generated biofilm and plaque, reduce gingivitis, inhibit tooth decay and formation of cavities, and/or reduce dentinal hypersensitivity. In some embodiments, said use is performed on a patient in need thereof, e.g., a patient demonstrated to have one or more of A. viscous, L. casei, S. oralis, V. parvula and F. nucleatum present in their oral cavities, or a patient diagnosed with an oral bacterial disease or disorder, e.g., an oral bacterial infection caused by one or more of A. viscous, L. casei, S. oralis, V. parvula and F. nucleatum.

In particular embodiments, Use 1 comprises the use of any of one of the oral care compositions according to Composition 1 or 1.1-1.45. In particular embodiments, Use 1 is further comprises:
1.1 Use 1, wherein the metal-amino acid complex is a zinc(II)-amino acid complex or a copper(II)-amino acid complex.
1.2 Use 1 or 1.1, wherein the amino acid is a basic amino acid.
1.3 Any foregoing use, wherein the amino acid is selected from lysine, glycine, trimethylglycine and arginine.
1.4 Any foregoing use, wherein the metal-amino acid complex further comprises a halide, e.g., a chloride.
1.5 Any foregoing use, wherein the complex is a zinc-lysine-chloride complex or a zinc-arginine chloride complex.
1.6 Any foregoing use, wherein the complex is a zinc lysine chloride complex having the chemical structure [Zn(C₆H₁₄N₂O₂)₂Cl]⁺Cl⁻, either in solution of the cationic cation (e.g., [Zn(C₆H₁₄N₂O₂)₂Cl]⁺) and the chloride anion, or in solid salt form, optionally in mono- or dihydrate form.
1.7 Any foregoing use, wherein the amount of zinc is 0.05-4% by weight.
1.8 Any foregoing use, wherein the composition is in the form of a toothpaste, gel, mouthwash, powder, cream, strip, or gum.
1.9 Any foregoing use, wherein the composition is in the form of a mouthwash.
1.10 Any foregoing use, wherein the use is effective in treating diseases, disorders or conditions of the oral cavity (e.g., gingivitis, periodontitis, halitosis, cavity formation, enamel erosion, or oral infection) or in disrupting the formation of plaque and bacterial biofilm.

The oral care composition used in the present disclosure can be in the form of any oral care formulations, including a toothpaste, gel, mouthwash, powder, cream, strip, gum, or any other known in the art. In some embodiments, the oral care composition used in the present disclosure is a mouthwash.

In another aspect, the present disclosure provides the use of a metal-amino acid complex, e.g., a zinc-amino acid complex or a copper-amino acid complex, in the manufacture of an oral care composition for use in Method 1 or Method 2, e.g., for use in promoting the aggregation of oral bacteria and/or promoting the immune clearance of oral bacteria. Such use may be beneficial for the treatment and/or prevention of a disease or disorder of the oral cavity, as described elsewhere herein, caused by said oral bacteria. Such metal-amino acid complexes include those described herein throughout, such as, zinc-amino acid complexes, zinc-amino acid-halide complexes, zinc-lysine-halide complexes, zinc-arginine halide complexes, zinc-lysine-chloride complexes and zinc-arginine-chloride complexes. In particular embodiments such complexes are the zinc-lysine-chloride complexes referred to as ZLC herein, e.g., a zinc lysine chloride complex having the chemical structure [Zn(C₆H₁₄N₂O₂)₂Cl]⁺Cl⁻, either in solution of the cation (e.g., [Zn(C₆H₁₄N₂O₂)₂Cl]⁺) and the chloride anion, or in solid salt form, e.g., crystal form, optionally in mono- or dihydrate form. Thus, for example, the present disclosure provides for the use of ZLC, e.g., a zinc lysine chloride complex having the chemical structure [Zn(C₆H₁₄N₂O₂)₂Cl]⁺Cl⁻, in the manufacture of an oral care composition for use in Method 1 or Method 2, e.g., for use in promoting the aggregation of oral bacteria and/or promoting the immune clearance of oral bacteria.

In another aspect, the present disclosure also provides the use of a metal-amino acid complex, e.g., a zinc-amino acid complex or a copper-amino acid complex, for use. Such use may be beneficial for the treatment and/or prevention of a disease or disorder of the oral cavity, as described elsewhere herein, caused by said oral bacteria. The present disclosure also provides the use of a metal-amino acid complex, e.g., a zinc-amino acid complex, for use in promoting the aggregation of oral bacteria and/or promoting the immune clearance of oral bacteria, according to Method 1 or Method 2. Such metal-amino acid complexes include those described herein throughout, such as, zinc-amino acid complexes, zinc-amino acid-halide complexes, zinc-lysine-halide complexes, zinc-arginine halide complexes, zinc-lysine-chloride complexes and zinc-arginine-chloride complexes. In particular embodiments such complexes are the zinc-lysine-chloride complexes referred to as ZLC herein, e.g., a zinc lysine chloride complex having the chemical structure [Zn(C₆H₁₄N₂O₂)₂Cl]⁺Cl⁻, either in solution of the cationic cation (e.g., [Zn(C₆H₁₄N₂O₂)₂Cl]⁺) and the chloride anion, or in solid salt form. e.g., crystal form, optionally in mono- or dihydrate form. Thus, for example, the present disclosure provides for the use of ZLC, e.g., a zinc lysine chloride complex having the chemical structure [Zn(C₆H₁₄N₂O₂)₂Cl]⁺Cl⁻, for use in promoting the aggregation of oral bacteria and/or promoting the immune clearance of oral bacteria, for example, according to Method 1 or Method 2.

In some embodiments, the methods and uses disclosed herein (Method 1, Method 2, and Use 1), may further comprise the administration of an antibacterial agent to provide a synergistic increase in antibacterial effect. This synergistic effect may arise from the ability of the metal amino-acid complex to promote bacterial aggregation and biofilm disruption, thus increasing the ability of the antibacterial agent to access the bacterial cells. Any antibacterial agent disclosed herein may be useful for said synergistic effect, including but not limited to, quaternary ammonium compounds (e.g., cetyl pyridinium chloride), halogenated diphenyl ethers (e.g., triclosan), and magnolia extracts (e.g., magnolol, honokiol).

The oral care composition used in the present disclosure can include the metal-amino acid complex or can include precursors thereof. For example, where the metal-amino acid complex is a zinc-amino acid-halide complex, the precursors which can react in situ with water to form the zinc amino acid halide complex include: (i) zinc and an amino acid hydrohalide, or (ii) zinc chloride and amino acid, or (iii) a zinc ion source, an amino acid, and a halogen acid, or (iv) combinations of (i), (ii), and/or (iii). In one embodiment, the zinc amino acid halide can be prepared at room temperature by mixing the precursors in a solution, such as water. The in situ formation provides ease of formulation. The precursors can be used instead of first having to form the zinc amino acid halide. In another embodiment, the water permitting formation of the zinc amino acid halide from the precursor comes from saliva and/or rinsing water that comes into contact with the composition after application.

Examples of amino acids useful in the metal-amino acid complexes of the oral care compositions used in the present disclosure include, but are not limited to, the common natural amino acids, e.g.: lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, and glutamic acid. In some embodiments the amino acid is a neutral or acidic amino acid, e.g., glycine. In some embodiments, the amino acid is an alkylated amino acid, e.g., a tri-N-alkylated amino acid, such as trimethylglycine.

The ability of the metal-amino acid complexes to promote the aggregation of oral bacteria, e.g., the zinc-amino acid complexes, is most notable when the complex is formed from a basic amino acid. By “basic amino acid” is meant the naturally occurring basic amino acids, such as arginine, lysine, and histidine, as well as any basic amino acid having a carboxyl group and an amino group in the molecule, which is water-soluble and provides an aqueous solution with a pH of about 7 or greater. Accordingly, basic amino acids include, but are not limited to, arginine, lysine, citrulline, ornithine, creatine, histidine, diaminobutanoic acid, diaminopropionic acid, salts thereof or combinations thereof. In certain embodiments, the amino acid is lysine. In other embodiments, the amino acid is arginine.

The halide may be chlorine, bromine, or iodine, most typically chlorine. The acid addition salt of an amino acid and a halogen acid (e.g., HCl, HBr, or HI) is sometimes referred to herein as an amino acid hydrohalide. Thus one example of an amino acid hydrohalide is lysine hydrochloride. Another is glycine hydrochloride.

In certain embodiments, the amount of zinc amino acid halide in the oral care composition used in the present disclosure is 0.05 to 10% by weight of the composition. In certain embodiments, precursors, e.g., zinc and amino acid hydrohalide, are present in amounts such that when combined into the zinc amino acid halide, the zinc amino acid halide would be present in an amount of 0.05 to 10% by weight of the composition. In either of these embodiments, the amount of the zinc amino acid halide can be varied for the desired purpose, such as a dentifrice or a mouthwash. In other embodiments, the amount of the zinc amino acid halide is at least 0.1, at least 0.2, at least 0.3, at least 0.4, at least 0.5, at least 1, at least 2, at least 3, or at least 4 up to 10% by weight of the composition. In other embodiments, the amount of the zinc amino acid halide is less than 9, less than 8, less than 7, less than 6, less than 5, less than 4, less than 3, less than 2, less than 1, less than 0.5 to 0.05% by weight of the composition. In other embodiments, the amounts are 0.05 to 5%, 0.05 to 4%, 0.05 to 3%, 0.05 to 2%, 0.1 to 5%, 0.1 to 4%, 0.1 to 3%, 0.1 to 2%, 0.5 to 5%, 0.5 to 4%, 0.5 to 3%, or 0.5 to 2% by weight of the composition.

In certain embodiments, zinc is present in the oral care composition used in the present disclosure in an amount of 0.05 to 10% by weight of the composition. In other embodiments, the amount of zinc is at least 0.1, at least 0.2, at least 0.3, at least 0.4, at least 0.5, at least 1, at least 2, at least 3, or at least 4 up to 10% by weight of the composition. In other embodiments, the amount of the zinc is less than 9, less than 8, less than 7, less than 6, less than 5, less than 4, less than 3, less than 2, less than 1, less than 0.5 to 0.05% by weight of the composition. In other embodiments, the amounts are 0.05 to 5%, 0.05 to 4%, 0.05 to 3%, 0.05 to 2%, 0.1 to 5%, 0.1 to 4%, 0.1 to 3%, 0.1 to 2%, 0.5 to 5%, 0.5 to 4%, 0.5 to 3%, or 0.5 to 2% by weight of the composition.

In certain embodiments, amino acid hydrohalide is present in the oral care composition used in the present disclosure in an amount of 0.05 to 30% by weight. In other embodiments, the amount is at least 0.1, at least 0.2, at least 0.3, at least 0.4, at least 0.5, at least 1, at least 2, at least 3, at least 4, at least 5, at least 10, at least 15, at least 20 up to 30% by weight. In other embodiments, the amount is less than 30, less than 25, less than 20, less than 15, less than 10, less than 5, less than 4, less than 3, less than 2, or less than 1 down to 0.05% by weight of the composition.

Where precursor materials are present in the oral care composition used in the present disclosure, they are preferably present in molar ratios approximately as required to produce the desired zinc amino acid halide, although an excess of one material or another may be desirable in certain formulations, e.g., to balance pH against other formulation constituents, to provide additional antibacterial zinc, or to provide amino acid buffer. Preferably, however, the amount of halide is limited, as constraining the level of halide somewhat encourages interaction between the zinc and the amino acid.

In some embodiments, the total amount of zinc in the oral care composition used in the present disclosure is 0.05 to 8% by weight of the composition. In other embodiments, the total amount of zinc is at least 0.1, at least 0.2, at least 0.3, at least 0.4, at least 0.5, or at least 1 up to 8% by weight of the composition. In other embodiments, the total amount of zinc in the composition is less than 5, less than 4, less than 3, less than 2, or less than 1 to 0.05% by weight of the composition.

In certain embodiments, a molar ratio of zinc to amino acid in the oral care composition used in the present disclosure is at least 2:1. In other embodiments, the molar ratio is at least 1:1, at least 1:2, at least 1:3, at least 1:4, 2:1 to 1:4, 1:1 to 1:4, 1:2 to 1:4, 1:3 to 1:4, 2:1 to 1:3, 2:1 to 1:2, 2:1 to 1:1, or 1:3. Above 1:4, it is expected that the zinc will be totally dissolved.

In certain embodiments, the oral care composition used in the present disclosure is anhydrous. By anhydrous, there is less than 5% by weight water, optionally less than 4, less than 3, less than 2, less than 1, less than 0.5, less than 0.1 down to 0% by weight water.

The carrier in the oral care composition used in the present disclosure represents all other materials in the composition other than the zinc amino acid halide complex or its precursors. The amount of carrier is then the amount to reach 100% by adding to the weight of the zinc amino acid halide, including any precursors.

Active Agents:

The oral care composition used in the present disclosure may comprise various agents which are active to protect and enhance the strength and integrity of the enamel and tooth structure and/or to reduce bacteria and associated tooth decay and/or gum disease, including or in addition to the zinc-amino acid-halide complexes. Effective concentration of the active ingredients used herein will depend on the particular agent and the delivery system used. It is understood that a toothpaste for example will typically be diluted with water upon use, while a mouth rinse typically will not be. Thus, an effective concentration of active in a toothpaste will ordinarily be 5-15× higher than required for a mouth rinse. The concentration will also depend on the exact salt or polymer selected. For example, where the active agent is provided in salt form, the counterion will affect the weight of the salt, so that if the counterion is heavier, more salt by weight will be required to provide the same concentration of active ion in the final product. Arginine, where present, may be present at levels from, e.g., about 0.1 to about 20 wt % (expressed as weight of free base), e.g., about 1 to about 10 wt % for a consumer toothpaste or about 7 to about 20 wt % for a professional or prescription treatment product. Fluoride where present may be present at levels of, e.g., about 25 to about 25,000 ppm, for example about 750 to about 2,000 ppm for a consumer toothpaste, or about 2,000 to about 25,000 ppm for a professional or prescription treatment product. Levels of antibacterial agents will vary similarly, with levels used in toothpaste being e.g., about 5 to about 15 times greater than used in mouthrinse. For example, a triclosan toothpaste may contain about 0.3 wt % triclosan.

Fluoride Ion Source:

The oral care composition used in the present disclosure may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al. Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. In certain embodiments, the oral care composition described herein may also contain a source of fluoride ions or fluorine-providing ingredient in amounts sufficient to supply about 25 ppm to about 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A toothpaste for general consumer use would typically have about 1000 to about 1500 ppm, with pediatric toothpaste having somewhat less. A dentifrice or coating for professional application could have as much as about 5,000 or even about 25,000 ppm fluoride. Fluoride ion sources may be added to the compositions described herein at a level of about 0.01 wt. % to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counterion in the salt.

Abrasives:

The oral care composition used in the present disclosure, e.g. Composition 1 et seq. may include silica abrasives, and may comprise additional abrasives, e.g., a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate; calcium carbonate abrasive; or abrasives such as sodium metaphosphate, potassium metaphosphate, aluminum silicate, calcined alumina, bentonite or other siliceous materials, or combinations thereof.

Other silica abrasive polishing materials useful herein, as well as the other abrasives, generally have an average particle size ranging between about 0.1 and about 30 microns, about between 5 and about 15 microns. The silica abrasives can be from precipitated silica or silica gels, such as the silica xerogels described in U.S. Pat. No. 3,538,230, to Pader et al. and U.S. Pat. No. 3,862,307, to Digiulio. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason. In certain embodiments, abrasive materials useful in the preparation of the oral care compositions described herein include silica gels and precipitated amorphous silica having an oil absorption value of less than about 100 cc/100 g silica and in the range of about 45 cc/100 g to about 70 cc/100 g silica. Oil absorption values are measured using the ASTA Rub-Out Method D281. In certain embodiments, the silicas are colloidal particles having an average particle size of about 3 microns to about 12 microns, and about 5 to about 10 microns. Low oil absorption silica abrasives particularly useful in the preparation of the compositions described herein are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of 29% by weight averaging about 7 to about 10 microns in diameter, and an oil absorption of less than about 70 cc/100 g of silica is an example of a low oil absorption silica abrasive useful in the practice of the present disclosure.

Foaming Agents:

The oral care composition used in the present disclosure also may include an agent to increase the amount of foam that is produced when the oral cavity is brushed. Illustrative examples of agents that increase the amount of foam include, but are not limited to polyoxyethylene and certain polymers including, but not limited to, alginate polymers. The polyoxyethylene may increase the amount of foam and the thickness of the foam generated by the oral care carrier component of the composition. Polyoxyethylene is also commonly known as polyethylene glycol (“PEG”) or polyethylene oxide. The polyoxyethylenes suitable for this composition will have a molecular weight of about 200,000 to about 7,000,000. In one embodiment the molecular weight will be about 600,000 to about 2,000,000 and in another embodiment about 800,000 to about 1,000,000. Polyox® is the trade name for the high molecular weight polyoxyethylene produced by Union Carbide. The polyoxyethylene may be present in an amount of about 1% to about 90%, in one embodiment about 5% to about 50% and in another embodiment about 10% to about 20% by weight of the oral care carrier component of the oral care compositions. Where present, the amount of foaming agent in the oral care composition (i.e., a single dose) is about 0.01 to about 0.9% by weight, about 0.05 to about 0.5% by weight, and in another embodiment about 0.1 to about 0.2% by weight.

Surfactants:

The oral care composition used in the present disclosure may contain anionic surfactants, for example:
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl sulfates, such as sodium lauryl sulfate,
iii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na).
iv. higher alkyl aryl sulfonates such as sodium dodecyl benzene sulfonate (sodium lauryl benzene sulfonate)
v. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

By “higher alkyl” is meant, e.g., C₁₋₃₀ alkyl. In particular embodiments, the anionic surfactant is selected from sodium lauryl sulfate and sodium ether lauryl sulfate. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. For example, concentrations used or a mouthwash are typically on the order of one tenth that used for a toothpaste. In one embodiment, the anionic surfactant is present in a toothpaste at from about 0.3% to about 4.5% by weight, e.g., about 1.5%. The oral care composition used in the present disclosure may optionally contain mixtures of surfactants, e.g., comprising anionic surfactants and other surfactants that may be anionic, cationic, zwitterionic or nonionic. Generally, surfactants are those which are reasonably stable throughout a wide pH range. Surfactants are described more fully, for example, in U.S. Pat. No. 3,959,458, to Agricola et al.; U.S. Pat. No. 3,937,807, to Haefele; and U.S. Pat. No. 4,051,234, to Gieske et al. In certain embodiments, the anionic surfactants useful herein include the water-soluble salts of alkyl sulfates having about 10 to about 18 carbon atoms in the alkyl radical and the water-soluble salts of sulfonated monoglycerides of fatty acids having about 10 to about 18 carbon atoms. Sodium lauryl sulfate, sodium lauroyl sarcosinate and sodium coconut monoglyceride sulfonates are examples of anionic surfactants of this type. In a particular embodiment, the compositions described herein, e.g., Composition 1, et seq., comprises sodium lauryl sulfate.

The surfactant or mixtures of compatible surfactants can be present in the compositions described herein in about 0.1% to about 5.0%, in another embodiment about 0.3% to about 3.0% and in another embodiment about 0.5% to about 2.0% by weight of the total composition.

Tartar Control Agents:

In various embodiments, the oral care composition used in the present disclosure may comprise an anticalculus (tartar control) agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. The composition thus may comprise phosphate salts. In particular embodiments, these salts are alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP) Na₅P₃O₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%. e.g., ca. 5-15%, by weight of the composition.

Polymers:

The oral care composition used in the present disclosure may also include additional polymers to adjust the viscosity of the formulation or enhance the solubility of other ingredients. Such additional polymers include polyethylene glycols, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts.

Silica thickeners, which form polymeric structures or gels in aqueous media, may be present in the oral care composition used in the present disclosure. Note that these silica thickeners are physically and functionally distinct from the particulate silica abrasives also present in the compositions, as the silica thickeners are very finely divided and provide little or no abrasive action. Other thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate can also be used as component of the thickening composition to further improve the composition's texture. In certain embodiments, thickening agents in an amount of about 0.5% to about 5.0% by weight of the total composition are used.

The oral care composition used in the present disclosure may include an anionic polymer, for example in an amount of from about 0.05 to about 5%. Such agents useful in compositions described herein are disclosed in U.S. Pat. Nos. 5,188,821 and 5,192,531; and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (M.W.) of about 30,000 to about 1,000,000, most preferably about 300,000 to about 800,000. These copolymers are available for example as Gantrez. e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805. The enhancing agents when present are present in amounts ranging from about 0.05 to about 3% by weight. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Water:

The oral care composition used in the present disclosure may comprise significant levels of water. Water employed in the preparation of commercial oral compositions should be deionized and free of organic impurities. The amount of water in the compositions includes the free water which is added plus that amount which is introduced with other materials.

Humectants:

In certain embodiments, it is also desirable to incorporate in the oral care composition used in the present disclosure a humectant to prevent the composition from hardening upon exposure to air. Certain humectants can also impart desirable sweetness or flavor to dentifrice compositions. Suitable humectants include edible polyhydric alcohols such as glycerine, sorbitol, xylitol, propylene glycol as well as other polyols and mixtures of these humectants. In some embodiments of the composition described herein, the principal humectant is glycerin, which may be present at levels of greater than 25%, e.g. 25-35% about 30%, with 5% or less of other humectants.

Unless stated otherwise, all percentages of composition components given in this specification are by weight based on a total composition or formulation weight of 100%.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### EXAMPLES

### Example 1

The general reaction for formation of zinc-lysine-chloride complex (ZLC) is as follows:

ZnO+2(Lysine-HCl)->[Zn(Lysine)₂Cl]Cl.2H₂O (ZLC)

A 2:1 molar ratio of ZnO:Lysine-HCl suspension is prepared with stirring at room temperature for about 12 hours. The mixture is centrifuged. 1 ml of supernatant is transferred into an NMR tube. The NMR tube is then placed in a closed test tube filled with ethanol for crystal growth. A number of colorless, cubic crystals are formed after a week. The crystal structure of ZLC crystal is determined by single crystal X-ray diffraction. The dimension of this complex molecule is 1.7 nm*7.8 nm*4.3 nm. In this complex, Zn cation is coordinated by two lysine ligands with two N atoms from NH₂ groups and O atoms from carboxylic groups in an equatorial plane. It displays a distorted square-pyramidal geometry with the apical position occupied by a Cl atom. This novel structure gives rise to a positive cation moiety, to which a Cl anion is combined to form an ionic salt.

Laboratory Scale-Up Synthesis of Pure ZLC Powder:

2 mole of Lysine HCl is dissolved in 1000 ml DI water with stirring at room temperature, 1 mole of solid ZnO is added slowly to the Lysine HCl solution with stirring and the stirring is continued at room temperature overnight (about 12 hours). The suspension solution is centrifuged at high speed for 15 mins. The supernatant is slowly poured into ethanol. A precipitate is formed immediately. Approximately 5-8 ml ethanol is needed to get g powder. The ethanol solvent with powder is filtered, and an off-white powder is obtained. The powder is placed in a 50° C. oven for drying and an 88% yield of product is obtained. PXRD confirms the purity of ZLC powder compared to ZLC crystal.

Low-pH Preparation of ZLC:

In an alternative method, lysine HCl (2 molar equivalents) is dissolved in deionized water with stirring at room temperature. Concentrated hydrochloric acid equal to approximately 0.5 molar equivalent is added to the reaction mixture. ZnO (1 molar equivalent) is then added to the reaction mixture, and the mixture is stirred until complete dissolution occurs, typically within a minutes to a few hours. The pH of the reaction is kept at 6 or below. The ZLC complex can be kept in solution in the final reaction mixture, which is clear, for more than 3 months at slightly acidic pH (i.e., <pH 7) without precipitation of ZnO. Alternatively, the reaction mixture can be spray dried to yield ZLC powder, or the reaction mixture can be adjusted to a near-neutral pH (e.g., 6.8), and the crystalline salt of ZLC can be isolated upon slow evaporation of the concentrated ZLC reaction mixture.

### Example 2

Presented in Tables 1A and 1B are exemplary mouthwash formulations containing ZLC as the zinc-amino acid complex.

TABLE 1A
Mouthwash with ZLC

| Ingredients | % |
|---|---|
| Sorbitol 70% sol | 5.5 |
| ZLC (2.53% Zn) | 40 |
| Na Saccharin | 0.02 |
| Propylene Glycol | 7 |
| Poloxomer 407 | 0.4 |
| Citric Acid | 0.02 |
| Potassium Sorbitol | 0.05 |
| Glycerin | 7.5 |
| Flavor | 0.1 |
| Deionized water | 39.41 |
| Total | 100 |
| Zn % | 1.01 |

TABLE 1B

| Ingredients | Weight % |
|---|---|
| Demineralized Water | 71.38 |
| Cocamidopropyl Betaine | 0.700 |
| Glycerin (99.0-101.0%) | 10.000 |
| Sorbitol | 10.000 |
| Propylene Glycol | 7.000 |
| Sodium Saccharin | 0.020 |
| Potassium Sorbate | 0.050 |
| Flavor | 0.100 |
| CP-4 (Spray Dried Powder) | 0.600 |
| Hydrochloric Acid (35%) | 0.149 |
| Total Components | 100.000 |
| Total Zinc % | 0.080 |

### Example 3

An oral gel toothpaste with ZLC as active ingredient is formulated. Zinc ion concentration is about 0.5% (w/w).

TABLE 2
Oral gel with ZLC (2.53% Zn)

| Ingredients | % |
|---|---|
| Sorbitol 70% sol | 76.03 |
| ZLC aqueous solution 2.53% Zn | 20.00 |
| Carboxymethyl cellulose (CMC) and | 0.70 |
| Trimethyl cellulose (TMC) | |
| Na Saccharin | 0.27 |
| Propylene Glycol | 3.00 |
| Total | 100.00 |
| % Zn | 0.506 |

### Example 4

Exemplary dentifrice comprising zinc-lysine, 1450 ppm fluoride, and phosphates is prepared as described in Table 3:

TABLE 3

| Ingredient | Wt % |
|---|---|
| PEG600 | 3 |
| CMC-7 | 0.65 |
| Xanthan | 0.2 |
| Sorbitol | 27 |
| Glycerin | 20 |
| Saccharin | 0.3 |
| Tetrasodium pyrophosphate | 0.5 |
| Calcium pyrophosphate | 0.25 |
| Sodium phosphate dibasic | 3.5 |
| Sodium fluoride | 0.32 |
| (to provide 1450 ppm fluoride) | |
| Titanium dioxide | 0.5 |
| Abrasive silica | 8 |
| Thickener silica | 8 |
| ZLC | 7 |
| Sodium lauryl sulfate | 1.5 |
| Flavoring | 1.2 |
| Water | QS |

### Example 5

Aggregation of Oral Bacteria:

Stimulated whole saliva from a human volunteer was harvested and mixed either with an untreated control (water), metal salt solutions (0.2% zinc oxide or 0.1% zinc oxide/0.05% zinc citrate mixture), amino acid solutions (0.3% arginine, 1% arginine, or 0.94% lysine), or metal-amino acid complex solutions (1.2% zinc-lysine complex, 1.33% zinc-arginine complex, or 1% copper-lysine complex) as described in the present disclosure. The solutions were incubated for 15 minutes at room temperature in culture dish wells, then photographs were taken. For the metal salts, each dish showed only undissolved and/or precipitated metal salt. For the amino acids, no change was observed compared to the negative control. Large aggregates of material slowly formed and settled to the bottom of the dishes for each metal-amino acid complex well. These results were confirmed both visually and under low-power magnification.

The above described experiment was repeated using round bottom test tubes instead of culture dishes. Photographs of each tube were made at 5-minute intervals for 30 minutes during incubation. These experiments confirmed a time-dependent formation of large aggregates only in the solution containing the metal-amino acid complexes.

To confirm that the aggregation phenomenon observed was due to bacterial aggregation, as opposed to host cell-mediated aggregation, the human saliva sample was first filter-sterilized (0.2 micron vacuum filter) to remove host cells, cellular debris, oral bacteria and large proteins. To this saliva sample was added a 5-species mix of common oral bacteria (A. viscous, L. casei, S. oralis, V. parvula and F. nucleatum). The same aggregation phenomenon was observed in this experiment as was observed for the whole saliva experiment.

To evaluate the aggregation quantitatively, Streptococcus oralis bacteria was suspended in sterile 0.9% NaCl medium in capped culture tubes. To each tube, a different test sample was added, and the absorbance of the medium at 610 nM was monitored over a period of about one hour. Bacteria of this size will remain suspended in solution for several hours before settling out under gravity. This gives such a solution a uniform haziness or opacity that can be quantified by measuring the absorbance of the solution at 610 nm. The aggregation of bacteria results in clumps of bacterial matter, which initially raises absorbance, followed by a sharp drop in absorbance as the bacterial matter settles out, leaving an increasingly transparent solution. Therefore, a decrease in absorbance signals the formation of bacterial aggregates. Results are shown in Table 4 below:

| | OD₆₁₀: | | | | | |
|---|---|---|---|---|---|---|
| | 1 | 9 | 21 | 28 | 40 | 55 |
| | minute | minutes | minutes | minutes | minutes | minutes |
| Bacteria only | 0.514 | 0.530 | 0.513 | 0.512 | 0.512 | 0.505 |
| 1.5% Arginine | 0.492 | 0.473 | 0.455 | 0.449 | 0.441 | 0.428 |
| 5% Arginine | 0.496 | 0.468 | 0.450 | 0.446 | 0.440 | 0.435 |
| 0.94% Lysine | 0.537 | 0.547 | 0.552 | 0.553 | 0.555 | 0.555 |
| 0.35% ZnCl₂ | 0.572 | 0.584 | 0.615 | 0.680 | 0.652 | 0.397 |
| 1.2% Zn-Lys | 0.549 | 0.551 | 0.574 | 0.515 | 0.357 | 0.182 |
| 1.33% Zn-Arg | 0.563 | 0.568 | 0.575 | 0.620 | 0.616 | 0.361 |

The results obtained show that the addition to the culture of a test solution containing no additive, 1.5% arginine, 5% arginine, 0.94% lysine, or 0.35% zinc chloride showed no appreciable change in OD (610 nm) or a rise in OD (610 nm) over the course of the experiment. In contrast, 1.33% zinc-arginine complex as additive results in a brief rise in absorbance which peaks at about 28 minutes, followed by a significant drop in absorbance by about 55 minutes. Similarly, the use of 1.2% zinc-lysine complex results in a peak of absorbance at about 21 minutes, followed by a significant drop by about 55 minutes. These results demonstrate the clear ability of metal-amino acid complexes to promote the rapid aggregation of oral bacteria. Even more pronounced results are obtained using A. viscous bacteria, and these are shown in Table 5 below. Only in the case of using zinc-lysine or zinc-arginine complex, the final test result is nearly zero absorbance at 610 nm:

| | OD₆₁₀: | | | | |
|---|---|---|---|---|---|
| | 1 | 14 | 27 | 46 | 66 |
| | minute | minutes | minutes | minutes | minutes |
| Bacteria only | 0.494 | 0.495 | 0.488 | 0.484 | 0.472 |
| 1.5% Arginine | 0.438 | 0.432 | 0.427 | 0.418 | 0.424 |
| 5% Arginine | 0.341 | 0.334 | 0.335 | 0.333 | 0.333 |
| 0.94% Lysine | 0.416 | 0.413 | 0.411 | 0.410 | 0.409 |
| 0.35% ZnCl₂ | 0.315 | 0.315 | 0.316 | 0.391 | 0.255 |
| 1.2% Zn-Lysine | 0.454 | 0.554 | 0.175 | 0.032 | 0.014 |
| 1.33% Zn-Arg | 0.436 | 0.474 | 0.623 | 0.369 | 0.025 |

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the scope of the invention should be construed broadly as set forth in the appended claims.

1. A method of promoting the aggregation and/or immune clearance of oral bacteria, the method comprising the step of administering to the oral cavity of a person an oral care composition comprising a metal-amino acid complex;
wherein said person has demonstrated one or more of A. viscous, L. casei, S. oralis, V. parvula and F. nucleatum present in their oral cavities.

2. The method of claim 1, wherein the metal-amino acid complex is a zinc(II)-amino acid complex or a copper(II)-amino acid complex.

3. The method of claim 1, wherein the amino acid is a basic amino acid.

4. The method of claim 1, wherein the amino acid is selected from lysine, glycine, trimethylglycine and arginine.

5. The method of claim 1, wherein the metal-amino acid complex further comprises a halide.

6. The method of claim 1, wherein the complex is a zinc-lysine-chloride complex or a zinc-arginine chloride complex.

7. The method of claim 1, wherein the complex is a zinc lysine chloride complex having the chemical structure [Zn(C6H14N2O2)2Cl]⁺Cl⁻, either in solution of the cationic cation and the chloride anion, or in solid salt form, optionally in mono- or dihydrate form.

8. The method of claim 6, wherein the amount of zinc is 0.05-4% by weight.

9. The method of claim 1, wherein the composition is in the form of a toothpaste, gel, mouthwash, powder, cream, strip, or gum.

10. The method of claim 1, wherein the method is effective in treating diseases, disorders or conditions of the oral cavity or in disrupting the formation of plaque and bacterial biofilm.

11. A method of treating a disease, disorder, or condition of the oral cavity, comprising the step of administering to a patient in need thereof, an oral care composition comprising a metal-amino acid complex, to promote the aggregation and/or immune clearance of oral bacteria;
wherein said patient has demonstrated one or more of A. viscous, L. casei, S. oralis, V. parvula and F. nucleatum present in their oral cavities.

12. The method of claim 11, wherein the metal-amino acid complex is a zinc(II)-amino acid complex or a copper(II)-amino acid complex.

13. The method of claim 11, wherein the amino acid is a basic amino acid.

14. The method of claim 11, wherein the amino acid is selected from lysine, glycine, trimethylglycine and arginine.

15. The method of claim 11, wherein the metal-amino acid complex further comprises a halide.

16. The method of claim 11, wherein the complex is a zinc-lysine-chloride complex or a zinc-arginine chloride complex.

17. The method of claim 11, wherein the complex is a zinc lysine chloride complex having the chemical structure [Zn(C6H14N2O2)2Cl]⁺Cl⁻, either in solution of the cationic cation and the chloride anion, or in solid salt form, optionally in mono- or dihydrate form.

18. The method of claim 17, wherein the amount of zinc is 0.05-4% by weight.

19. The method of claim 11, wherein the composition is in the form of a toothpaste, gel, mouthwash, powder, cream, strip, or gum.

20. The method of claim 11, wherein the disease, disorder or conditions of the oral cavity is selected from gingivitis, periodontitis, halitosis, cavity formation, enamel erosion, or an oral infection.

21. The method of claim 1, wherein said person is recovering from a treatment which predisposes the person to an oral bacterial infection.
